(19) United States
(12) Patent Application Publication
(10) Pub. No.: US 2008/0009487 A1
(43) Pub. Date: Jan. 10, 2008

(54) Treatment of athersclerotic disease

(76) Inventor: Andrew Sternlicht, Chestnut Hill, MA (US)

Correspondence Address: CLARK & ELBING LLP, 101 FEDERAL STREET, BOSTON, MA, 02110, US

(21) Appl. No.: 11/712,656

(22) Filed: Mar. 1, 2007

Related U.S. Application Data
(60) Provisional application No. 60/779,274, filed on Mar. 3, 2006.

(51) Int. Cl. A61K 31/5383; A61P 9/00

(52) U.S. Cl. 514/229.5

(57) ABSTRACT

The invention features a method of inhibiting the progression of intima-media thickening, or reducing the intima-media thickness (IMT) in arteries in a patient in need thereof by administering to the patient a rifamycin in an amount effective to inhibit the progression of intima-media thickening, or reduce the IMT. The invention also features a method for treating or preventing cerebral vascular disease in a patient in need thereof by administering to the patient a rifamycin in an amount effective to treat the cerebral vascular disease in the patient.

### CROSS-REFERENCE TO RELATED APPLICATIONS

This application claims benefit from U.S. Provisional Application No. 60/779,274, filed Mar. 3, 2006, which is hereby incorporated by reference.

### BACKGROUND OF THE INVENTION

Atherosclerosis and its complications lead to half of all adult deaths in the United States and other western societies, and its incidence is increasing in developing countries. Atherosclerotic disease causes intima-media thickening and plaque formation encroaching the arterial lumen, decreasing the effective luminal radius of afflicted arterial segments, and producing an anatomic and sometimes functional obstruction to blood flow. When these conditions arise, an increase in vascular resistance can lead to a reduction in distal perfusion pressure and blood flow.

Evidence suggesting that atherosclerosis is a chronic inflammatory disease has led to considerable research into the role played by infectious agents. Specific pathogens detected in atherosclerotic lesions could act as additional factors in accelerating the disease progression. Ott and co-workers report a wide diversity (>50 clones) of bacterial DNA found in atherosclerotic lesions of all samples of coronary heart disease (CHD) patients (Circulation 113:929-937, 2006). Representative pathogens found in these atherosclerotic lesions include C. pneumoniae, C. trachomatis, Staphylococcus species, Streptococcus species, Psuedomonas species, and the Sphingobacterium species. The high overall diversity of bacterial DNA found suggests that there are multiple bacterial colonizations of atherosclerotic lesions. While the role of bacterial infections in atherosclerotic lesions is not completely understood, it is generally thought that these bacterial infections contribute to the progression of atherosclerotic disease in some way, whether by participating or promoting aspects of atherogenesis in conjunction with conventional triggers, by acceleration of atherosclerosis, or by driving an inflammatory response (Katz and Shannon, Circulation 113:920-922, 2006). To date, Chlamydia pneumoniae shows the strongest association with the progression of atherosclerotic disease.

Chlamydia (C.) pneumoniae is an obligate intracellular prokaryotic pathogen and is a common causative pathogen of many acute upper and lower respiratory tract infections, which are often self-limiting and subclinical. C. pneumoniae can infect and survive in a wider range of host cell types, such as lung epithelium, resident macrophages, circulating monocytes, arterial smooth muscle cells, and vascular endothelium. Since exposure to C. pneumoniae is extremely common, infections occur repeatedly throughout life for most people. Antibiotic treatment for C. pneumoniae infection can be difficult as the life cycle includes resident time in morphologic forms not susceptible to most antibiotic therapy.

High-resolution B-mode ultrasonography has been proved to be a valid and reliable method of detecting initial structural atherosclerotic changes in the arterial wall. Increased intima-media thickening of the common carotid artery (CCA) is a validated marker and a powerful predicator for the occurrence of subsequent atherosclertosic clinical events.

### SUMMARY OF THE INVENTION

In general, the present invention is based on the observation that treatment with rifalazil resulted in reduced C. pneumoniae burden and plaque area stenosis in an animal model of atherosclerosis in which C. pneumoniae infection exacerbated plaque deposition, compared with placebo-treated animals. Based on this observation, we believe that rifalazil and other rifamycins are useful for the treatment of cerebral vascular disease (CVD) and atherosclerosis, and, by extension, also useful for inhibiting the progression of intima-media thickening, or reducing the intima-media thickness (IMT) of arterial walls.

Accordingly, the invention features a method of inhibiting the progression of intima-media thickening, or reducing the IMT in arteries in a patient in need thereof by administering to a patient a rifamycin in an amount effective to inhibit the progression of intima-media thickening or reduce the IMT. In one embodiment, the patient has not been diagnosed as having a bacterial infection that can be treated by administration of a rifamycin. In another embodiment, the patient has been diagnosed as having an infection of C. pneumoniae. In another embodiment, the patient is seropositive for C. pneumoniae.

A patient is considered to be treated if any one of the following exemplary conditions achieves significant improvement: (1) reduction of arterial wall inflammation, as measured, for example, by an improvement in arterial wall distensibility, (2) inhibition of the progression of intima-media thickening, or (3) reduction of the intima-media thickness (IMT).

The invention also features a method of treating arterial vasculature, and thereby treating or preventing CVD, in a patient in need thereof (i.e., a patient diagnosed as having CVD or at risk for developing CVD) by administering to the patient a rifamycin in an amount effective to treat CVD in the patient.

The invention also features a method for reducing the frequency of occurrence of cerebral vascular events in a patient at risk for such events by administering to patients a rifamycin in an amount effective to reducing the frequency of occurrence of cerebral vascular events.

The invention also features methods for:
(i) reducing the number and/or frequency of occurrence of cerebral vascular events;
(ii) reducing the functional impairment associated with the progression of atherosclerosis;
(iii) reducing localized inflammation in an atherosclerotic plaque;
(iv) reducing the size of an atherosclerotic plaque;
(v) improving arterial distensibility;
(vi) preserving arterial luminal diameter;
(vii) reducing the non-calcified component of plaque;
(viii) changing the overall density spectral analysis of atherosclerotic plaque in order to reduce the risk of subsequent cerebral vascular events;
(ix) reducing levels of inflammatory or risk-predictive biomarkers (e.g., C-reactive protein, IL-6, IL-11, lipoprotein-associated phospholipase A2, fractalkine, monocyte chemotactic protein 1, neopterin, tumor necrosis factor receptors I and II, selectin, fibrinogen, ICAM-1, VCAM-1, myeloperoxidase);
(x) reducing vascular smooth muscle cell proliferation and/or the cellular and molecular products of vascular smooth muscle cell proliferation (including those mediated by the Toll-Like Receptor-2 pathways (see Yang et al. Arterioscler. Thromb. Vasc. Biol., 25: 2308-2314, 2005)); and/or
(xi) improving endothelial function and capability in a patient.

Each of these methods involves administering an effective amount of a rifamycin (i.e., an amount sufficient to achieve the desired result).

In one embodiment of any of the foregoing methods, the patient has been diagnosed as having cerebral vascular disease. In another embodiment, the patient has been diagnosed with coronary arterial disease or a disease of the aortic, renal, mesenteric, pulmonary, hepatic, periosteal, or ophthalmic arteries. In another embodiment, the patient has not been diagnosed as having a bacterial infection that can be treated by administration of a rifamycin. In another embodiment, the patient has been diagnosed as having an infection of C. pneumoniae, C. trachomatis, Streptococcus spp., or Staphlococcus spp. In another embodiment, the patient is seropositive for C. pneumoniae (for example, the patient has an IgG antibody titer ≧1:64, as determined by a microimmunofluorescence assay). In yet another embodiment, the patient is seronegative for C. pneumoniae.

In any of the foregoing methods, a preferred rifamycin is rifalazil. The dosage of rifalazil normally ranges between 0.001 mg to 100 mg, preferably is 1-50 mg, or more preferably 2-25 mg. The rifalazil may be given daily (e.g., a single oral dose of 0.001 mg to 100 mg/day, preferably 2.5 to 25 mg/day) or less frequently (e.g., a single oral dose of 5 mg/week, 12.5 mg/week, or 25 mg/week). Treatment may be given for a period of one day to one year, or longer. In one embodiment, the rifalazil is administered once per week in an amount of between 12.5 and 25 mg/week for 4-20 weeks. This protocol may be repeated periodically (e.g., every 3, 6, 12, or 36 months) for up to the lifetime of the patient. In another embodiment, a rifamycin is administered at an initial dose of 2.5 mg to 100 mg for one to seven consecutive days, followed by a maintenance dose of 0.005 mg to 10 mg once every one to seven days for one month, one year, or even for the life of the patient. In another embodiment, a rifamycin is administered at an initial dose of 2.5 to 100 mg once a week, for a period of two to 16 weeks, followed by a dose of 2.5 to 50 mg once a week, once each two weeks, once a month, or once each two months, for a period of months to years, or even for the remaining lifespan of a patient.

The rifamycin can be a rifamycin other than rifalazil. For example, the rifamycin may be, without limitation, rifampin, rifabutin, rifapentin, or rifaximin administered in a dosage that normally ranges between 50 to 1000 mg/day. These rifamycins may be given daily (e.g., a single oral dose of 50 to 600 mg/day) or less frequently (e.g., a single oral dose of 50, 100, or 300 mg/week). Treatment may be administered for a period of one day to one year, or even longer. In one embodiment, one of these rifamycins is administered at an initial dose of 600 mg to 2000 mg for one to seven consecutive days, followed by a maintenance dose of 100 mg to 600 mg once every one to seven days for one month, one year, or even for the life of the patient.

If desired, a rifamycin may be administered in conjunction with one or more additional agents such as anti-inflammatory agents, e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) steroids (e.g., cortisone, dexamethasone, hydrocortisone, methylprednisolone, prednisolone, prednisone, triamcinolone), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), ticlopidine, clopidogrel, angiotensin converting enzyme inhibitors, beta blockers, pentoxifylline, cilostazol, estrogen replacement therapy, lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These secondary therapeutic agents may be administered within 14 days, 7 days, 1 day, 12 hours, or 1 hour of administration of a rifamycin, or simultaneously therewith. The additional therapeutic agents may be present in the same or different pharmaceutical compositions as the rifamycin of the invention. When present in different pharmaceutical compositions, different routes of administration may be used. For example, rifalazil may be administered orally, while a second agent may be administered by intravenous, intramuscular, or subcutaneous injection.

By “rifalazil” is meant:

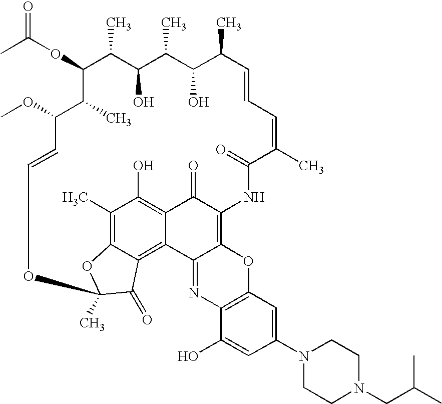

By “atherosclerotic disease” or “atherosclerosis” is meant a chronic, progressive disease in which plaques made up of cholesterol deposits, calcium, and abnormal cells develop on the inner lining of the arteries, arterioles, as well as veins that have been surgically moved to function as arteries, and result in the narrowing or obstruction of the blood vessel by plaque, and the development of atherosclerosis-related diseases.

By “atherosclerotic plaque” or “atheromatous plaque” is meant a waxy deposit consisting of smooth muscle cells, immune cells (e.g., lymphocytes, macrophages, or monocytes), lipid products (e.g., lipoproteins, or cholesterol), cellular waste products, and calcium, fibrous tissue, or other substances within the inner lining of an artery, or a vascular lesion that develops in the vessel wall, and in late stages may suddenly rupture, and reduce or totally stop blood flow in the lumen (stenosis), leading to damage of the tissue downstream which has lost needed blood flow.

By “cerebral vascular disease” or “CVD” is meant any abnormality of the cerebral vessels (e.g., the left and right internal carotid; the left and right vertebral arteries; the middle cerebral artery; the superior cerebellar artery; the posterior inferior cerebellar artery; the basilar artery; and the anterior communicating, anterior cerebral, internal carotid, posterior communicating and posterior cerebral arteries which form the vascular network known as the Circle of Willis) resulting from a pathologic process of the blood vessels, e.g. occlusion of the lumen by a thrombus or embolus, rupture of the vessel, any lesion or altered permeability of the vessel wall, and increased viscosity or other change in quality of blood. Disorders of cerebral circulation include any diseases of the vascular system that causes ischemia or infarction of the brain or spontaneous hemorrhage into the brain or subarachnoid space.

By “cerebral vascular event” is meant a sudden neurological deficit in the brain caused by a lack of blood supply and oxygen to the brain. Cerebral vascular events include embolic stroke, thrombotic stroke, hemorrhagic stroke, transient ischemic attack (TIA), and reversible ischemic neurologic deficit (RIND).

By “ischemic stroke” is meant a cerebral vascular event caused by atherosclerosis, embolism, or microangiopathy (small artery disease, the occlusion of small cerebral vessels). Ischemic stroke is commonly divided into thrombotic and embolic stroke.

By “embolic stroke” is meant a cerebral vascular event caused by an embolus, or a traveling particle in a blood vessel, which flows with the bloodstream into progressively smaller arteries until it becomes lodged, inhibiting passage of blood. An embolus is most frequently a blood clot, but it can also be a plaque broken off from an atherosclerotic blood vessel.

By “thrombotic stroke” is meant a cerebral vascular event caused by occlusion of an artery by the buildup of a thrombus, or clot.

By “hemorrhagic stroke” is meant a cerebral vascular event caused by a burst blood vessel in the brain.

By “transient ischemic attack” or “TIA” is meant a cerebral vascular event characterized by a sudden-onset severe headache (“thunderclap headache”), and an acute loss of focal cerebral function with symptoms lasting from 5 minutes to several hours, which then resolve completely.

By “reversible ischemic neurologic deficit” or “RIND” is meant a mild ischemic stroke with no persisting neurological disability. The symptoms last for more than 24 hours and typically resolve within three weeks.

By “functional impairment” is meant impairment of bodily function resulting from atherosclerotic disease, or the perceived impairment of ability to carry out functional tasks of everyday activities. Examples of functional impairment include numbness or weakness of the body and especially numbness or weakness on one side of the body (hemiplegia), paralysis, trouble speaking (aphasia), trouble understanding speech, trouble seeing in one eye or (rarely) both eyes, pupils of unequal size, impaired swallowing reflex, trouble walking, dizziness, loss of balance or coordination, loss of consciousness, severe headache or “thunderclap headache”, inability to perform learned movements (apraxia), or arteriosclerotic dementia. Perceived functional impairment can be measured, for example, using a questionnaire such as the SF-36.

By “arteriosclerotic dementia” is meant deterioration in a previously normal intellect and/or memory due to repeated clinical or subclinical episodes of cerebral vascular events.

By “arterial stenosis” is meant a condition in which blood flow is restricted in the artery wall by the formation of plaques, causing the vessel to become narrow and stiff.

By “intima-media thickness” or “IMT” is meant the combined thickness of the intima and media portions of the arterial wall. Increased IMT is a marker for stiffening of the arteries and atherosclerosis. “Reducing the IMT” is meant reducing the combined thickness of the intima and media portions of the arterial wall by at least 10%, 20%, 30%, 40%, 50%, 60%, 70%, 80%, 90%, 95%, or 99%.

By “intima-media thickening” is meant a thickening of the intima and media portions of the arterial wall. “Inhibiting the progression of intima-media thickening” is meant halting or reducing the progression of thickening of the intima and media portions of the arterial wall by at least 10%, 20%, 30%, 40%, 50%, 60%, 70%, 80%, 90%, 95%, or 99%.

In general, by “reducing” or “inhibiting” progression, impairment, an event, frequency, or number is meant reducing or inhibiting by at least 10%, 20%, 30%, 40%, 50%, 60%, 70%, 80%, 90%, 95%, or 99%.

By “arterial distensibility” is meant the elasticity, of an artery. Arterial distensibility can be measured by non-invasive ultrasonography (wall movement detector system). Reduced arterial distensibility is a marker for arterial inflammation, stiffening of the arteries and atherosclerosis.

By “arterial luminal diameter” is meant the inner diameter, from wall to wall, of an artery.

By “seropositive” is meant a positive serum reaction test for the presence of antibodies in a blood sample.

By “coronary arterial disease or “CAD” is meant a condition caused by obstruction of the coronary arteries, such as atherosclerotic plaques or intima media thickening resulting in diminished blood flow under certain conditions.

By “angina” is meant the symptoms a patient experiences any time the heart muscle is not getting enough blood flow through the coronary arteries. Angina is usually perceived as a discomfort, often a pressure-like pain, in or around the chest, shoulders, neck or arms.

By “stable angina” is meant angina that occurs in a nearly predictable fashion. Stable angina generally indicates that a stable atherosclerotic plaque is present in one of the coronary arteries, causing a partial obstruction of that artery. When the patient is at rest, the partially obstructed artery is able to meet the needs of the cardiac muscle. But when exertion occurs (or some other stress that causes increased cardiac work,) the obstruction prevents an adequate increase in blood flow to the muscle, and angina is experienced. Thus, stable angina implies a fixed, stable atherosclerotic plaque.

By “unstable angina” is meant angina that occurs sporadically, or unpredictably, or at rest. There is no particular pattern to unstable angina. Unstable angina implies the existence of an unstable plaque, one that has partially ruptured, or in which blood clots may be forming and breaking off, so that the lumen of the artery is not fixed, but is changing.

By “inflammatory or risk-predictive biomarkers” is meant a levels of certain chemicals, proteins, or metabolic by products produced by the body under conditions of stress or inflammation. These may be used to stratify for a given level of inflammation or the inflammatory response. Such a tool may be used to assess relative risk for subsequent clinical events in atherosclerotic disease, which is typically accompanied by arterial inflammation. Examples of biomarkers include C-reactive protein, myeloperoxidase, F-2 urinary isoprostanes, IL-6, IL-11, lipoprotein-associated phospholipase A2, fractalkine, monocyte chemotactic protein 1, neopterin, tumor necrosis factor receptors I and II, selectin, fibrinogen, ICAM-1, and VCAM-1.

By “treat” or “treating” is meant monitoring in a patient (1) the reduction of arterial wall inflammation, as measured, for example, by an improvement in arterial wall distensibility; (2) the inhibition of the progression of intima-media thickening, or (3) the reduction of the intima-media thickness (IMT). A patient is considered treated if any one of the aforementioned conditions achieves significant improvement.

By “a patient in need thereof” is meant a patient diagnosed with a particular disease, disorder or condition, or at risk of developing a particular disease, disorder or condition.

### DETAILED DESCRIPTION OF THE INVENTION

The invention features a method for inhibiting the progression of intima-media thickening, or reducing the intima-media thickness (IMT) of arteries in a patient in need thereof by administering to a patient a rifamycin in an amount effective to inhibit progression of intima-media thickening, or reduce IMT. Additionally, the invention features a method for treating arterial vasculature, and thereby treating or preventing cerebral vascular disease (CVD), in a patient in need thereof by administering to a patient a rifamycin in an amount effective to treat CVD in a patient. The invention also features a method of administering rifamycin to a patient having been diagnosed with atherosclerosis, CVD, coronary arterial disease (CAD), or a disease of the aortic, renal, mesenteric, pulmonary, hepatic, periosteal, or ophthalmic arteries. CVD is an abnormality of the cerebral vessels resulting from a pathologic process of the blood vessels, e.g., occlusion of the lumen by a thrombus or embolus, rupture of the vessel, any lesion or altered permeability of the vessel wall, and increased viscosity or other change in quality of blood. CAD is a condition caused by the narrowing of the coronary arteries that supply blood and oxygen to the heart.

The most common cause of arterial disease is atherosclerosis. Atherosclerosis is a chronic, progressive disease in which plaques develop on the inner lining of the arteries, and result in the narrowing or obstruction of the blood vessel. The atherosclerotic plaque, a deposit of smooth muscle cells, immune cells, lipid products, cellular waste products, and calcium, fibrous tissue, or other substances within the inner lining of an artery, may suddenly rupture, and reduce or totally stop blood flow in the lumen (stenosis), leading to damage of tissue downstream which has lost needed blood flow. Loss of blood flow and oxygen supply to the brain, for example from blockage of a blood vessel from a thrombus or embolus, can lead to cerebral vascular events, such as embolic stroke, thrombotic stroke, hemorrhagic stroke, transient ischemic attack (TIA), or reversible ischemic neurologic deficit (RIND). Likewise, loss of blood flow and oxygen supply to the coronary arteries can cause the heart to slow or stop (heart attack or myocardial infarction), can cause symptoms such as chest pain (stable or unstable angina), shortness of breath, and/or acute embolic or thrombotic episodes.

Rifamycins

Rifamycins can be employed in any of the methods of the invention. Rifamycins are compounds characterized by a chromophoric naphtha-hydroquinone group spanned by an aliphatic bridge. Exemplary rifamycins are rifalazil (3′-hydroxy-5′-(4-isobutyl-1-piperazinyl)benzoxazinorifamycin; also known as KRM-1648 or ABI-1648), rifampin, rifabutin, rifapentin, and rifaximin. Other rifamycins are disclosed in U.S. Pat. Nos. 4,690,919; 4,983,602; 5,786,349; 5,981,522; 6,316,433 and 4,859,661, U.S. Patent Application Nos. 60/341,130 and 60/341,591, and U.S. Patent Publication Nos. US2005-0043298 A1; US2005-0137189 A1; and US2005-0197333 A1, each of which is hereby incorporated by reference.

The structure of rifalazil is shown below.

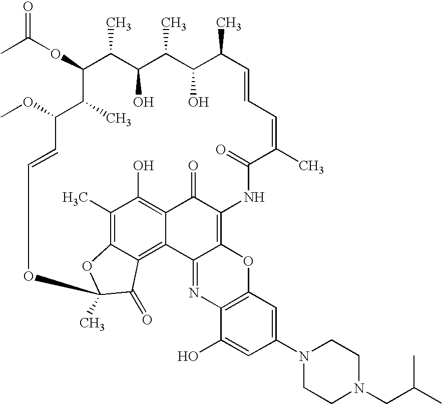

Rifalazil is a dark blue solid that is partially amorphous and partially crystalline. There is no observable melting point and no polymorphs have been detected.

Rifalazil is a highly lipophilic molecule having limited solubility in water at physiological pH (approximately 200 ng/mL). Evidence of the highly lipophilic behavior of the molecule is illustrated in the partition coefficient (n-octanol:water) of between 70, 569 and over 900,000 in different experiments (Log P range; 4.9-5.9).

Rifalazil degrades to a 25-desacetyl derivative under both acidic and basic conditions. Typical of ester hydrolysis, the degradation in highly alkaline solutions is rapid while at an acidic pH, e.g., pH 1, the degradation at room temperature is slower, approximately 6% in one hour.

The drug product currently being produced is a hard gelatin capsule containing rifalazil that has been formulated using microgranules made as described in U.S. Pat. No. 5,547,683. Materials Processing Technology Inc. (Patterson, N.J.) manufactures the granulated rifalazil which is subsequently encapsulated at ProClinical Pharmaceutical Services (Phoenixville, Pa.). This formulation for the 25 mg rifalazil capsules is summarized in Table 1, below.

TABLE 1

| | | 25 mg |
|---|---|---|
| | | Strength |
| | | Amount per |
| | | Dosage Unit |
| Ingredient | Function | (mg) |
| Rifalazil | Active substance | 25.00 |
| Mannitol, USP | Binder & Filler | 106.93 |
| Colloidal Silicon Dioxide, NF | Glidant | 0.63 |
| Hydroxypropyl Cellulose, USP | Binder, granulating agent | 0.89 |
| Water, USP | Solvent | 0.65 |
| Polysorbate 80, NF | Surfactant, wetting agent | 0.16 |
| Magnesium Stearate, NF | Lubricant | 0.67 |

| | |
|---|---|
| Total Capsule Fill Weight | 134.93 |

Use of Rifamycins in the Treatment of Cerebral Vascular Disease

Both preclinical and clinical lines of evidence support the use of rifalazil in the methods of the invention. First, in a rabbit model of atherosclerosis in which C. pneumoniae infection exacerbated plaque deposition and atherosclerotic changes in the arterial wall, treatment with rifalazil resulted in reduced C. pneumoniae burden and plaque area stenosis compared with placebo-treated animals. Second, the clinical efficacy of rifalazil in eradication of Chlamydia infection has been successfully demonstrated in a Phase 2 study in men with non-gonococcal urethritis in which a single oral dose eradicated Chlamydia in >86% of patients. Third, rifalazil is 2000 times more potent against C. pneumoniae than roxithromycin, a potent anti-chlamydial agent currently registered in European countries. In addition, based on overall safety data in animals and man, we believe that a dose of rifalazil can safely be administered, tolerated, and will likely result in clinical benefit through its anti-Chlamydia action in these patients.

Inhibition and Regression of Intima-Media Thickening

The invention features a method of inhibiting the progression of intima-media thickening, or reducing the IMT in arteries in a patient in need thereof by administering a rifamycin in an amount effective to inhibit the progression of intima-media thickening, or reduce the IMT. The progression of intima-media thickening can be determined by measuring the IMT, i.e. the combined thickness of the intima and media portions of the carotid or femoral arterial wall, at various times. IMT is a non-invasive, quick, safe, accurate, FDA approved, ultrasound measurement and is a reliable marker for generalized atherosclerosis on the basis of the positive association between IMT and the severity of several different cardiovascular disease risk factors, coronary artery disease, heart attacks, and cerebral vascular diseases such as stroke. Increased IMT is a validated marker for stiffening of the arteries and atherosclerosis. Lowered IMT progression rates and absolute thickness of IMT have been associated with reduced frequency and number of occurrence of cerebral vascular events, as well as reduced frequency of occurrence of unstable angina, myocardial infarction, hospitalization for such, sudden cardiac death, and peripheral arterial revascularization (Taylor et al., Circulation 110:23 3512-3517, 2004). Thus, the invention also features a method for reducing the frequency of occurrence of cerebral vascular events in patients at risk for such events by administering to said patients a rifamycin in an amount effective to inhibit the progression of intima-media thickening, or reduce the IMT.

The invention also features a method for reducing impairment of bodily function resulting from atherosclerotic disease, or the perceived impairment of ability to carry out functional tasks of everyday activities. Examples of functional impairment include numbness or weakness of the body and especially numbness or weakness on one side of the body (hemiplegia), paralysis, trouble speaking (aphasia), trouble understanding speech, trouble seeing in one eye or (rarely) both eyes, pupils of unequal size, impaired swallowing reflex, trouble walking, dizziness, loss of balance or coordination, loss of consciousness, severe headache or “thunderclap headache”, inability to perform learned movements (apraxia), or arteriosclerotic dementia. Perceived functional impairment can be measured, for example, using a questionnaire such as the SF-36.

Additionally, the invention features a method for administering to the patient with atherosclerosis a rifamycin in an amount effective to eradicate or reduce localized inflammation of the arterial walls, to reduce the overall size of the atherosclerotic plaque, and to reduce the dangerous “soft” plaque (the non-calcified component of plaque) most responsible for embolic and thrombotic stroke. Reduction of these symptoms should, in turn, lead to improved arterial distensibility, or elasticity of the arteries, increased arterial luminal diameter, and reduce the risk of subsequent cerebral vascular events.

Atherosclerosis and cerebral vascular disease are generally accompanied by an inflammatory response. Measurement of inflammatory biomarkers, such as C-reactive protein (CRP) levels, can give some indication as to the progression or seriousness of the disease. Other inflammatory biomarkers include myeloperoxidase, F-2 urinary isoprostanes, IL-6, IL-11, lipoprotein-associated phospholipase A2, fractalkine, monocyte chemotactic protein 1, neopterin, tumor necrosis factor receptors I and II, selectin, fibrinogen, ICAM-1, and VCAM-1. Thus, the invention features a method for reducing the levels of inflammatory or risk-predictive biomarkers in a patient having cerebral vascular disease by administering to the patient a rifamycin in an amount effective to reduce the levels of the inflammatory or risk-predictive biomarkers.

The invention features a method of reducing vascular smooth muscle cell proliferation and/or the cellular and molecular products of vascular smooth muscle cell proliferation in a patient having cerebral vascular disease by administering to a patient an effective amount of a rifamycin. The invention also features a method of improving endothelial function and capability in a patient having cerebral vascular disease by administering to a patient an effective amount of a rifamycin.

The invention also features a method of administering to a patient a rifamycin and one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) steroids (e.g., cortisone, dexamethasone, hydrocortisone, methylprednisolone, prednisolone, prednisone, triamcinolone), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), ticlopidine, clopidogrel, angiotensin converting enzyme inhibitors, beta blockers, pentoxifylline, cilostazol, estrogen replacement therapy, lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin) or HDL-C raising drugs or other cholesterol esterase transfer protein (CETP) inhibitors (e.g., torcetrapib). These additional agents can be administered simultaneously, or within 14 days, 7 days, 1 day, 12 hours, 1 hour of administration of a rifamycin.

Animal Studies in Atherosclerosis

A study in rabbits demonstrated that rifalazil has effects on the acceleration of atherosclerosis induced by chlamydial infection in a rabbit model fed a high cholesterol diet.

Forty-five rabbits were fed a modestly enhanced (25%) cholesterol diet. Thirty received three separate C. pneumoniae inoculations performed at 3-week intervals. Similarly, 15 control rabbits were intranasally inoculated with 1 mL of normal saline under the same conditions. Three days after final inoculation, rabbits were assigned to treatment groups as shown in Table 2.

TABLE 2

| Inoculate | (n) | Treatment Group |
|---|---|---|
| C. pneumoniae | 10 | Azithromycin, 30 mg/kg PO daily for 1 week; |
| | | 30 mg/kg PO twice weekly for 6 weeks |
| C. pneumoniae | 10 | Rifalazil, 5 mg/kg PO daily for 1 week; |
| | | 5 mg/kg PO twice weekly for 6 weeks |
| C. pneumoniae | 10 | Placebo, 1 mL normal saline/kg PO daily for |
| | | 1 week; 1 mL/kg PO twice weekly for 6 weeks |
| Normal saline | 5 | Azithromycin, 30 mg/kg PO daily for 1 week; |
| | | 30 mg/kg PO twice weekly for 6 weeks |
| Normal saline | 5 | Rifalazil, 5 mg/kg PO daily for 1 week; |
| | | 5 mg/kg PO twice weekly for 6 weeks |
| Normal saline | 5 | Placebo, 1 mL normal saline/kg PO daily for |
| | | 1 week; 1 mL/kg PO twice weekly for 6 weeks |

Nineteen weeks after beginning antibiotic treatment, the rabbits were euthanized. After euthanasia, hearts were examined histologically and by immunofluorescence. Animal serum samples were also examined for seropositivity to the infecting organism.

In a similar study published previously, C. pneumoniae-infected rabbits accumulated significantly more plaque than uninfected rabbits, and azithromycin treatment had a significant effect in preventing Chlamydia-induced plaque formation (Muhlestein, 2000). In the current study, the time after infection and treatment with either azithromycin or rifalazil was increased by seven weeks, a more rigorous test for the durability of treatment. Consistent with previously published work, C. pneumoniae-infected rabbits accumulated more plaque than non-infected rabbits (p=0.08). Rifalazil treatment reduced the infectious burden of Chlamydia in the vasculature, (p<0.001) as did azithromycin (p=0.005). In addition, rifalazil (p=0.08), but not azithromycin (p=0.94), showed a trend in producing significantly reduced plaque area stenosis compared with placebo-treated animals (Table 3).

TABLE 3

| | | Plaque Area | P value versus infected |
|---|---|---|---|
| Treatment Group | (n) | Stenosis (%) | placebo |
| Infected, placebo-treated | 10 | 29.0 | |
| Uninfected animals | 10 | 22.8 | p = 0.08 |
| Infected, azithromycin- | 10 | 30.2 | p = 0.94 |
| treated | | | |
| Infected, rifalazil-treated | 10 | 24.2 | p = 0.13 |

### OTHER EMBODIMENTS

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, or related fields are intended to be within the scope of the invention.

All publications mentioned in this specification are herein incorporated by reference to the same extent as if each independent publication was specifically and individually incorporated by reference.

What is claimed is:

1. A method of inhibiting the progression of intima-media thickening, or reducing the intima-media thickness (IMT) in arteries in a patient in need thereof by administering to said patient a rifamycin in an amount effective to inhibit said progression of intima-media thickening, or reduce said IMT.

2. A method of claim 1, in which said intima-media thickening and said IMT refer to intima-media thickening and IMT of the carotid artery.

3. The method of claim 1, wherein said patient has been diagnosed as having coronary arterial disease or a disease of the aortic, renal, mesenteric, pulmonary, hepatic, periosteal, or ophthalmic arteries.

4. A method for treating or preventing cerebral vascular disease in a patient in need thereof by administering to said patient a rifamycin in an amount effective to treat said cerebral vascular disease in said patient.

5. The method of any one of claims 1 or 4, wherein said rifamycin is rifalazil.

6. The method of claim 5, wherein said rifalazil is administered to said patient in an amount of 12.5 to 50 mg, at a frequency of once per week for 4-20 weeks.

7. The method of claim 6, wherein said rifalazil is administered to said patient in an amount of 12.5 to 25 mg, at a frequency of once per week for 4-20 weeks.

8. The method of claim 6, wherein said rifalazil is administered to said patient in an amount of 12.5 to 25 mg, at a frequency of once per week for 12 weeks.

9. The method of claim 6, wherein said rifalazil is administered to said patient in an amount of 12.5 to 50 mg, at a frequency of once per week for 8-16 weeks.

10. The method of claim 5, wherein said rifalazil is administered at an initial dose of 2.5 to 100 mg once a week, for a period of two to 16 weeks, followed by a dose of 2.5 to 50 mg once a week, once each two weeks, once a month, or once each two months, for a period of at least six months and up to the lifetime of said patient.
